(19) United States
(12) Patent Application Publication
(10) Pub. No.: US 2009/0012179 A1
(43) Pub. Date: Jan. 8, 2009

(54) Use of Tyrphostins to Stimulate Neurite Outgrowth

(76) Inventors: Chunhe Wang, Portland, OR (US); Bruce G. Gold, West Linn, OR (US); Kim A. Neve, Portland, OR (US); Yong-Ping Zhong, Beaverton, OR (US); Xiaolin Yu, Portland, OR (US); Robert A. Johnson, Portland, OR (US)

Correspondence Address: KLARQUIST SPARKMAN, LLP, 121 SW SALMON STREET, SUITE 1600, PORTLAND, OR, 97204, US

(21) Appl. No.: 11/658,618

(22) PCT Filed: Aug. 4, 2005
(86) PCT No.: PCT/US05/28027

§ 371 Date: Jan. 25, 2007

Related U.S. Application Data
(60) Provisional application No. 60/599,350, filed on Aug. 5, 2004.

(51) Int. Cl. A61K 31/05; C12N 5/06; A61P 25/00

(52) U.S. Cl. 514/646; 435/375

(57) ABSTRACT

Disclosed herein are several methods for promoting nerve regeneration. The methods include administering at least one tyrphostin, or a pharmaceutically acceptable salt thereof. In one non-limiting example, the tyrphostin is tyrphostin A9. These methods can be used to promote nerve growth in the central or peripheral nervous system. The methods are of use in treating neurodegenerative diseases, such as Parkinson's disease, or in treating the transaction of a partial or complete transaction of a nerve in the peripheral or central nervous system.

### PRIORITY

This claims the benefit of U.S. Provisional Application No. 60/599,350, filed Aug. 5, 2004, which is incorporated by reference herein in its entirety.

### STATEMENT OF GOVERNMENT SUPPORT

This invention was made with United States government support pursuant to grant MH45372, from the National Institutes of Health, and pursuant to funding from the Department of Veteran's Affairs; the United States government has certain rights in the invention.

### FIELD

This application relates to compounds and agents useful in the treatment of neurological injury and disease.

### BACKGROUND

Following traumatic or mechanically induced axonal degeneration in the peripheral nervous system, axonal regeneration often ensues, resulting in functional recovery. However, the rate of axonal elongation (3-4 mm/day) is slow, and sometimes does not result in recovery of full neurological function. If neurological function is restored, recovery usually occurs in weeks or months, depending upon the distance between the site of injury and the target tissue. Therapies that speed regeneration over long distances would be highly beneficial to patients and would significantly reduce health care costs.

Other neurological conditions result from dysfunction of neurons in the peripheral or central nervous systems that is caused by chronic disease or injury. Chronic disease processes, including autoimmune and degenerative disorders, can permanently and progressively damage the nervous system, and (particularly in the central nervous system) usually results in permanent loss of function. Such loss of neurological function is a major cause of physical incapacitation and death throughout the world.

Neurons in the central and peripheral nervous systems degenerate as a normal function of human development and aging. Pathological neuron degeneration, however, is a serious condition seen in several neurological disorders. Neuronal degeneration can be specific or diffuse, and can lead to sensory, motor, and cognitive impairments. Neurodegenerative disorders encompass a range of seriously debilitating conditions including Parkinson disease, amyotrophic lateral sclerosis (ALS, “Lou Gehrig's disease”), multiple sclerosis, Huntington disease, Alzheimer disease, Pantothenate kinase associated neurodegeneration (PKAN, formerly Hallervorden-Spatz syndrome), multiple system atrophy, diabetic retinopathy, multi-infarct dementia, macular degeneration, and the like. These conditions are characterized by a gradual but relentless worsening of the patient's condition over time. These disorders affect a large population of humans, especially older adults.

Many advances have been made in years past in gaining a better understanding of Parkinson disease, Alzheimer disease, and Huntington disease. The primary cause of cognitive dysfunction for all three disorders has been directly linked to neuron degeneration, usually in specific areas of the brain. Parkinson disease is linked to degeneration of neurons in the substantia nigra, while Alzheimer disease is in some part due to loss of pyramidal neurons in the limbic cortex (Braak, E. & Braak, H., 1999, In: V. E. Koliatsos & R. R. Ratan (eds.), Cell Death and Diseases of the Nervous System, Totowa, N.J.: Humana Press, pp. 497-508). Huntington disease's cognitive deficits are produced by degeneration of cells in the caudate nucleus of the striatum. However, although the symptoms and progression of these diseases are well characterized, there is a need for compounds that can be used to treat these disorders.

Many of the compounds previously shown to stimulate nerve regeneration have undesired side-effects, such as immunosuppression (FK506 and analogs that retain immunosuppressant activity) or androgenic or estrogenic stimulation. There is therefore a need to provide a class of nerve growth stimulating compounds that are well tolerated by subjects who take them.

### SUMMARY

Disclosed herein are several methods for promoting nerve regeneration. These methods include administering at least one tyrphostin, or a pharmaceutically acceptable salt thereof, such as tyrphostin A9. These methods can be used to promote nerve growth in the central or peripheral nervous system.

Methods are described for promoting nerve regeneration and/or neurite outgrowth in a subject. The method that includes administering to the subject a therapeutically effective amount of a composition that includes at least one tyrphostin or pharmaceutically acceptable salt thereof, such as tyrphostin A9. In one example, the subject has a degenerative disorder of the nervous system. In another embodiment, the subject has a partial or completely transected nerve. In additional examples, the subject has a partial transaction of the spinal cord or a peripheral nerve.

In one embodiment, a method is provided for inducing neurite outgrowth from a nerve cell that includes contacting the cell with an effective amount of a composition that includes at least one tyrphostin or pharmaceutically acceptable salt thereof, such as tyrphostin A9. The cell can be in vivo or in vitro. In a further example, the tyrphostin or the pharmaceutically acceptable salt thereof is administered in conjunction with nerve growth factor (NGF).

The foregoing and other features and advantages will become more apparent from the following detailed description of several embodiments, which proceeds with reference to the accompanying figures.

### BRIEF DESCRIPTION OF THE FIGURES

FIGS. 1A-B are bar graphs and digital images showing that tyrphostin A9 stimulated ERK activation in NS20Y neuroblastoma cells, but not in HEK293 transformed human embryonic kidney cells. The levels of ERK activation were quantified by immunoblotting for the dually phosphorylated ERK. Data=mean±SD, n=3.

FIGS. 2A-D are bar graphs and digital images showing that tyrphostin A9 stimulated robust and sustained ERK activation in a time and dose-dependent manner in NS20Y cells. FIG. 2A shows a time course of the activation of ERK by 1 μM A9. FIG. 2B shows ERK activation in response to treatment with various concentrations of A9 for 45 minutes. FIG. 2C shows the effect of different signaling inhibitors on A9 (1 μM, 45 minutes)-stimulated activation of ERK. FIG. 2D shows that tyrphostin A9 analogue tyrphostin 1, PDGF receptor inhibitors AG1295 and AG370, or NGF inhibitor AG 879 had little or no effect on ERK activation in NS20Y cells.

FIG. 3 is a bar graph and digital image showing the D2 receptor agonist quinpirole stimulated a short-lived ERK activation in NS20Y cells.

FIG. 4 is a set of digital images showing that tyrphostin A9 induced ERK phosphorylation in dopaminergic neurons. Primary cultures of rat embryonic SNc neurons were stimulated with vehicle (NT) or A9 (1 μM) for 1 hour and then stained for tyrosine hydroxylase (TH) and phospho-ERK (p-ERK). Images were digitally captured with an Olympus CX640 fluorescence microscope.

FIGS. 5A-C is a set of graphs and digital images showing that tyrphostin A9 stimulated neurite elongation in SH-SY5Y cells. Data shown are representative of three experiments. FIG. 5A is a set of graphs. Left panels: Mean neurite lengths in SH-SY5Y cells at 96 and 168 hours shown for no treatment (NT), NGF only, NGF+FK506 (100 nM, a positive control), or NGF+A9 at different concentrations. Right panels: Cumulative histograms showing the distribution of the neurite length. The shift to the right for the A9-treated groups is more pronounced than that for the NGF-treated. FIG. 5B is a set of digital images of light micrographs showing representative SH-SY5Y cells treated with vehicle (left), 10 ng/ml of NGF (center), and 10 ng/ml of NGF+100 nM of A9 (right) for 168 hours. Magnification×120. FIG. 5C is a bar graph showing that the MEK inhibitor SL327 blocked A9-induced neurite outgrowth in SH-SY5Y cells.

FIGS. 6A-6B are a bar graph and a set of digital images showing that A9 treatment stimulated dopamine uptake increase (FIG. 6A) and neuroprotection against MPP⁺ (FIG. 6B) in primary SNc neurons cultures. Primary cultures of rat embryonic SNc neuron cultures were grown on treated 12-well plates (for dopamine uptake) or coverslips (for neuroprotection) for 10 days as described in the Examples section. In FIG. 6A, the neurons were treated with different concentrations of A9 for 6 days before dopamine uptake was measured. Data=mean±SD (n=3). In FIG. 6B, MPP⁺ (25 μM) was added into medium on day 11, while A9 was added either on day 10 (pretreatment) or day 12 (after MPP⁺). Half of the medium was changed with fresh A9 on day 14. The neurons were fixed on day 16 and stained for tyrosine hydroxylase (TH, green) and total-ERK (red). Images were captured with fluorescence microscopy at magnification×100.

FIG. 7 is a set of bar graphs showing a comparison of the effects of tyrphostin A9 with those of AG23 and AG82 in stimulation of ERK activation in NS20Y cells and neurite outgrowth in SH-SY5Y cells. For analysis of process length, cells (20 fields per well) were randomly photographed at 72 and 168 hours. Neurite lengths were measured on photographic prints using a SummaSketch III digitizing tablet connected with Bioquant Classic 95 software (R&M Biometrics, Nashville, Tenn.); only those processes greater than two times the cell body length were measured. Data from identically treated wells were not different and were therefore combined. Mean values and histograms were constructed from these data. Histograms were compared using a Mann-Whitney U test, which makes no assumptions about the shape of the distribution.

FIG. 8 is a digital image of sections stained using immunohistochemistry (see the Examples section). The results showed that A9 treatment preserves normal axons and reduces the presence of damaged axons in the CNS of SJL mice with EAE. The upper row shows the total axons stained by SMI312 (dark grey), while the lower row shows damaged axons stained by SMI32 (lower row). The sections were counterstained using hemotoxylin and eosin.

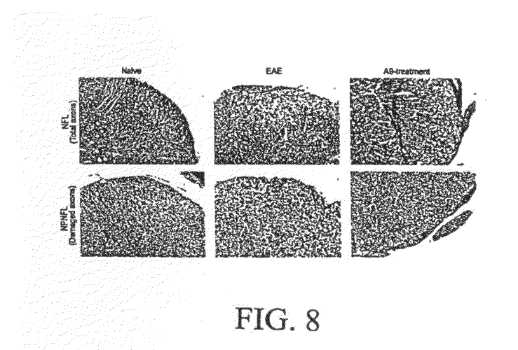

### DETAILED DESCRIPTION

### I. Abbreviations

ERK: extracellular signal-regulated protein kinase

IM: intramuscular

IP: intraperitoneal

IV: intravenous

HIV: human immunodeficiency virus

MAPK: mitogen-activated protein kinase

MS: multiple sclerosis

NGF: nerve growth factor

PBS: phosphate buffered saline

SQ: subcutaneous

TH: tyrosine hydroxylase

### II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of this disclosure, the following explanations of specific terms are provided:

### Chemical Terms

“Alkyl” refers to a cyclic, branched, or straight chain alkyl group containing only carbon and hydrogen, and unless otherwise mentioned, typically contains one to twelve carbon atoms. This term is further exemplified by groups such as methyl, ethyl, n-propyl, isobutyl, t-butyl, pentyl, pivalyl, heptyl, adamantyl, and cyclopentyl. Alkyl groups can either be unsubstituted or substituted with one or more substituents as described below.

“Substituted alkyl” refers to an alkyl as described above in which one or more hydrogen or carbon atom of the alkyl is replaced by another group such as a halogen, aryl, substituted aryl, cycloalkyl, substituted cycloalkyl, and combinations thereof. Illustrative substituted alkyls include benzyl, trichloromethyl, and the like.

“Heteroalkyl” refers to an alkyl as described above in which one or more hydrogen or carbon atom of the alkyl is replaced by a heteroatom such as N, O, P, B or S. An alkyl substituted with a heterocycloalkyl, substituted heterocycloalkyl, heteroaryl, substituted heteroaryl, alkoxy, aryloxy or amino is included within “heteroalkyl.” Illustrative heteroalkyls include cyano, benzoyl, 2-pyridyl, 2-furyl and the like.

“Cycloalkyl” refers to a saturated or unsaturated cyclic non-aromatic hydrocarbon radical having a single ring or multiple condensed rings. Illustrative cycloalkyls include cyclopentyl, cyclohexyl, bicyclooctyl and the like.

“Substituted cycloalkyl” refers to cycloalkyl as described above in which one or more hydrogen or carbon atom is replaced by another group such as a halogen, aryl, substituted aryl, alkoxy, aryloxy, amino and combinations thereof.

“Heterocycloalkyl” refers to a cycloalkyl radical as described above in which one or more of the carbon atoms of the cyclic radical is replaced by a heteroatom such as N, O, P, B or S. Illustrative heterocycloalkyls include, for example, piperazinyl, morpholinyl, tetrahydropyranyl, tetrahydrofuranyl, piperidinyl, pyrrolindinyl, oxazolinyl and the like.

“Substituted heterocycloalkyl” refers to a heterocycloalkyl radical as described above in which one or more hydrogen or carbon atom is replaced by another group such as a halogen, aryl, substituted aryl, alkoxy, aryloxy, amino and combinations thereof.

“Aryl” refers to an aromatic substituent that may be a single aromatic ring or multiple aromatic rings that are fused together, linked covalently, or linked to a common group such as a methylene or ethylene moiety. The common linking group may also be a carbonyl as in benzophenone or oxygen as in diphenylether or nitrogen in diphenylamine. The aromatic ring(s) may include phenyl, naphthyl, biphenyl, diphenylether, diphenylamine and benzophenone among others. In particular examples, aryls have between 1 and 20 carbon atoms.

“Substituted aryl” refers to an aryl radical as described above in which one or more hydrogen or carbon atom is replaced by one or more functional groups such as alkyl, substituted alkyl, cycloalkyl, substituted cycloalkyl, heterocycloalkyl, substituted heterocycloalkyl, halogen, alkylhalos, hydroxy, amino, alkoxy, and thio. Illustrative substituted aryls include chlorophenyl, 3,5-dimethylphenyl, 2,6-diisopropylphenyl and the like.

“Heteroaryl” refers to aromatic rings in which one or more carbon atoms of the aromatic ring(s) are replaced by a heteroatom(s) such as N, O, P, B or S. Heteroaryl refers to structures that may be a single aromatic ring, multiple aromatic rings or one or more aromatic rings coupled to one or more nonaromatic rings. Illustrative heteroaryls include, for example, thiophene, pyridine, isoxazole, phthalidimide, pyrazole, indole, furan and the like.

“Substituted heteroaryl” refers to a heteroaryl radical as described above in which one or more hydrogen or carbon atom is replaced by one or more functional groups such as alkyl, substituted alkyl, cycloalkyl, substituted cycloalkyl, heterocycloalkyl, substituted heterocycloalkyl, halogen, alkylhalos, hydroxy, amino, alkoxy, and thio.

“Alkoxy” refers to an —OZ radical wherein Z is selected from alkyl, substituted alkyl, cycloalkyl, substituted cycloalkyl, heterocycloalkyl, substituted heterocycloalkyl, and combinations thereof. Illustrative alkoxy radicals include methoxy, ethoxy, benzyloxy, and t-butoxy. A related term is “aryloxy” wherein Z is selected from aryl, substituted aryl, heteroaryl, substituted heteroaryl, and combinations thereof. Illustrative alkoxy radicals include phenoxy, substituted phenoxy, 2-pyridinoxy, 8-quinalinoxy and the like.

“Amino” refers to the group —NZ¹Z² wherein each of Z¹ and Z² is independently selected from hydrogen, alkyl, substituted alkyl, cycloalkyl, substituted cycloalkyl, heterocycloalkyl, substituted heterocycloalkyl, aryl, substituted aryl, heteroaryl, substituted heteroaryl, alkoxy, aryloxy, and combinations thereof.

“Thio” refers to the group —SZ¹Z² wherein each of Z¹ and Z² is independently selected from hydrogen, alkyl, substituted alkyl, cycloalkyl, substituted cycloalkyl, heterocycloalkyl, substituted heterocycloalkyl, aryl, substituted aryl, heteroaryl, substituted heteroaryl, alkoxy, aryloxy, and combinations thereof.

“Halogen” refers to fluoro, bromo, chloro and iodo substituents.

### Other Terms

“Administration of” and “administering” a compound or composition should be understood to mean providing a compound, a prodrug of a compound, or a pharmaceutical composition as described herein.

An “animal” is a living multicellular vertebrate organism, a category that includes, for example, mammals and birds. A “mammal” includes both human and non-human mammals. “Subject” includes both human and animal subjects.

“Axonal growth” or “axonal regeneration” as used herein refer both to the ability of an axon to grow and to the ability of an axon to sprout. An axon sprout is defined as a new process that extends from an existing or growing axon. (See, e.g., Ma et al., Nat. Neurosci. 2:24-30, 1999).

“Dosage” means the amount delivered in vivo to a subject of a compound, a prodrug of a compound, or a pharmaceutical composition as described herein. A “kinase” refers to an enzyme that catalyzes the transfer of a phosphate group from one molecule to another. A “serine threonine kinase” transfers phosphate groups to a hydroxyl group of serine and/or threonine in a polypeptide. A “phosphatidyl inositol 3-kinase” refers to an enzyme that phosphorylates inositol lipids at the D-3 position of the inositol ring to generate the 3-phosphoinositides, phosphatidylinositol 3-phosphate [PtdIns(3)P], phosphatidylinositol 3,4-bisphosphate [PtdIns(3,4)P₂] and phosphatidylinositol 3,4,5-trisphosphate [PtdIns(3,4,5)P₃]. GenBank Accession No. AAB53966 sets forth an exemplary amino acid sequence of the catalytic subunit of human phosphatidyl inositol 3-kinase. An “extracellular signal-regulated kinase” is a serine/threonine kinase including extracellular signal-regulated protein kinase 1 (ERK1) and ERK2. GenBank Accession No. NM_{—}002746 sets forth an exemplary ERK1, which is also called p44-MAPK or MAPK3. GenBank Accession No. NM_{—}138957 sets forth an exemplary ERK2, which is also called p42-MAPK or MAPK1.

ERK1 and ERK2 (or p44 and p42 MAPK) are activated by a wide variety of extracellular signals, and are the two best-studied members in the ubiquitous mitogen-activated protein kinase (MAPK) family of serine threonine kinases. The activation of ERK occurs through phosphorylation of threonine and tyrosine at the sequence TpEYp by the upstream MAPK/ERK kinase (MEK). ERK regulates a diverse array of functions, including cell growth and proliferation, differentiation, and apoptosis.

A “neurodegenerative disorder” is an abnormality in the nervous system of a subject, such as a mammal, in which neuronal integrity is threatened. Without being bound by theory, neuronal integrity can be threatened when neuronal cells display decreased survival or when the neurons can no longer propagate a signal. Specific, non-limiting examples of a neurodegenerative disorder are Alzheimer's disease, Pantothenate kinase associated neurodegeneration, Parkinson disease, Huntington disease (Dexter et al., Brain 114:1953-1975, 1991), HIV encephalopathy (Miszkziel et al., Magnetic Res. Imag. 15:1113-1119, 1997), and amyotrophic lateral sclerosis.

Alzheimer's disease manifests itself as pre-senile dementia. The disease is characterized by confusion, memory failure, disorientation, restlessness, speech disturbances, and hallucination in mammals (Medical, Nursing, and Allied Health Dictionary, 4th Ed., 1994, Editors: Anderson, Anderson, Glanze, St. Louis, Mosby).

“Multiple sclerosis” (MS) is a chronic, neurological, autoimmune, demyelinating disease. MS can cause blurred vision, unilateral vision loss (optic neuritis), loss of balance, poor coordination, slurred speech, tremors, numbness, extreme fatigue, changes in intellectual function (such as memory and concentration), muscular weakness, paresthesias, and blindness. Many subjects develop chronic progressive disabilities, but long periods of clinical stability may interrupt periods of deterioration. Neurological deficits may be permanent or evanescent.

The pathology of MS is characterized by an abnormal immune response directed against the central nervous system. In particular, T-lymphocytes are activated against the myelin sheath of the neurons of the central nervous system causing demyelination. Axons are also injured during the course of MS. In the demyelination process, myelin is destroyed and replaced by scars of hardened “sclerotic” tissue which is known as plaque. These lesions appear in scattered locations throughout the brain, optic nerve, and spinal cord. Demyelination interferes with conduction of nerve impulses, which produces the symptoms of multiple sclerosis. Most subjects recover clinically from individual bouts of demyelination, producing the classic remitting and exacerbating course of the most common form of the disease known as relapsing-remitting multiple sclerosis.

Parkinson disease is a slowly progressive, degenerative, neurologic disorder characterized by resting tremor, loss of postural reflexes, and muscle rigidity and weakness (Medical, Nursing, and Allied Health Dictionary, 4th Ed., 1994, Editors: Anderson, Anderson, Glanze, St. Louis, Mosby).

Amyotrophic lateral sclerosis is a degenerative disease of the motor neurons characterized by weakness and atrophy of the muscles of the hands, forearms, and legs, spreading to involve most of the body and face (Medical, Nursing, and Allied Health Dictionary, 4th Ed., 1994, Editors: Anderson, Anderson, Glanze, St. Louis, Mosby).

Pantothenate kinase associated neurodegeneration (PKAN, MIM# 234200, formerly Hallervorden-Spatz syndrome) is an autosomal recessive neurodegenerative disorder associated with brain iron accumulation. Clinical features include extrapyramidal dysfunction, onset in childhood, and a relentlessly progressive course (Dooling et al., Arch. Neurol. 30:7083, 1974). PKAN is a clinically heterogeneous group of disorders that includes classical disease with onset in the first two decades, dystonia, high globus pallidus iron with a characteristic radiographic appearance (Angelini et al., J. Neurol. 239:417-425, 1992), and often either pigmentary retinopathy or optic atrophy.

A “neurodegenerative-related disorder is a disorder such as speech disorders that are associated with a neurodegenerative disorder. Specific non-limiting examples of a neurodegenerative related disorders include, but are not limited to, palilalia, tachylalia, echolalia, gait disturbance, perseverative movements, bradykinesia, spasticity, rigidity, retinopathy, optic atrophy, dysarthria, and dementia.

“Nerve” encompasses a single bundle of nerve fibers or a plurality of bundles of nerve fibers.

“Nerve regeneration” refers to axonal regeneration and restoration of connectivity within neural networks after nerve injury or damage. For example, nerve regeneration can include complete axonal nerve regeneration, including vascularization and reformation of the myelin sheath. More specifically, when a nerve is severed, a gap is formed between the proximal and distal portions of the injured nerve. In order for the nerve axon to regenerate and reestablish nerve function, it must navigate and bridge the gap. Nerve regeneration involves the proximal end forming neurite growth cones that navigate the gap and enter endoneural tubes on the distal portion, thus re-connecting the neural network. Thus, a necessary action for nerve regeneration is sufficient neurite elongation as well as a sufficient rate of neurite elongation. In certain examples, the desirable neurite elongation is significantly greater than that achieved with nerve growth factor alone in cell cultures as described below. For instance, the neurite elongation may be at least about 200 μm, and more particularly about 200 μm to about 1000 μm, in treated cells at 168 hours. With respect to nerve regeneration in animals, a functional improvement may be observed, for example, with at least about a 15% increase in the rate of neurite elongation, more particularly at least about a 30% rate increase, relative to the rate of neurite elongation for untreated nerve injuries.

“Inhibiting” or “treating” a disease refers to inhibiting the full development of a disease or condition, for example, in a subject who is at risk for a disease such as an autoimmune disease, graft-versus-host disease, or rejection of a transplanted tissue or organ. “Treatment” refers to a therapeutic intervention that ameliorates a sign or symptom of a disease or pathological condition after it has begun to develop. As used herein, the term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease.

The “peripheral nervous system” (PNS) is the part of an animal's nervous system other than the Central Nervous System. Generally, the PNS is located in the peripheral parts of the body and includes cranial nerves, spinal nerves and their branches, and the autonomic nervous system. The “central nervous system” (CNS) is the part of the nervous system of an animal that contains a high concentration of cell bodies and synapses and is the main site of integration of nervous activity. In higher animals, the CNS generally refers to the brain and spinal cord.

A “pharmaceutical agent” or “drug” refers to a chemical compound or composition capable of inducing a desired therapeutic or prophylactic effect when properly administered to a subject.

“Pharmaceutically acceptable salts” include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide. These salts may be prepared by standard procedures, for example by reacting the free acid with a suitable organic or inorganic base. Any chemical compound recited in this specification may alternatively be administered as a pharmaceutically acceptable salt thereof. “Pharmaceutically acceptable salts” are also inclusive of the free acid, base, and zwitterionic forms. Descriptions of suitable pharmaceutically acceptable salts can be found in Handbook of Pharmaceutical Salts, Properties, Selection and Use, Wiley VCH (2002).

“Phosphorylation” refers to the creation of a phosphate derivative of an organic molecule (such as a protein or a lipid). In a cell, this can be achieved by transferring a phosphate group from adenosine triphosphate (ATP).

A “subject” is a living multi-cellular vertebrate organisms, a category that includes both human veterinary subjects, including human and non-human mammals.

“Therapeutically-active” refers to an agent, compound or composition capable of inducing a desired therapeutic or prophylactic effect when properly administered to a subject. In this case, the desired therapeutic effect is nerve regeneration.

“Therapeutically-effective amount” or “nerve regeneration promoting-amount” is an amount sufficient to achieve a statistically significant promotion of nerve cell regeneration compared to a control. Nerve cell regeneration can be readily assessed using an in vitro assay, such as the assays described in the Examples below. Alternatively, nerve regeneration can be determined in an in vivo assay or by direct or indirect signs of nerve cell regeneration in a subject. Preferably, the increase in nerve regeneration is at least 10%, preferably at least 30%, and most preferably 50% or more compared to a control.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### DETAILED DESCRIPTION OF SEVERAL EMBODIMENTS

Methods are disclosed herein for increasing neurite outgrowth in vivo and in vitro, and for promoting nerve regeneration in a subject. The methods include administering an effective amount of a tyrphostin, or a pharmaceutically acceptable salt thereof, which as a structure set forth below:

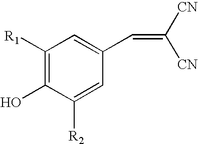

wherein R₁ and R₂ can be hydrogen (H) or can be any alkyl group, such as lower alkyl group. In one embodiment, at least one of R₁ and R₂ are a hydrogen (H). In another example, at least one of R₁ and R₂ are a lower alkyl group and include one to ten carbon atoms. In a further embodiment, both R₁ and R₂ include one to ten carbon atoms. In this embodiment, specific examples are R₁ and R₂ are methyl, ethyl, n-propyl, isopropyl, n-butyl, sec-butyl, t-butyl and isobutyl groups.

In one example, both R₁ and R₂ are t-butyl groups. Thus, the tyrphostin is tyrphostin A9, or a pharmaceutically acceptable salt thereof, which has the following chemical structure:

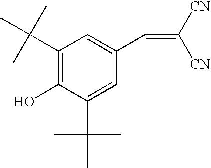

In one embodiment, the tyrphostin activates an ERK, such as ERK1 and/or ERK2. Activation of the ERK kinase can be measured by assays well known in the art. For example, Western blot technology is used with the cell proteins separated by electrophoresis and antibodies that bind to the substrate (such as the phosphorylated (activated) for of ERK) are utilized. Alternatively, the ability of immunoprecipitated kinase to phosphorylate a substrate such as myelin basic protein can be measured.

One of skill in the art can readily determine if a tyrphostin activates ERK. For example, cells are treated in vitro with test compounds at 37° C. in a 5% CO₂ humidified atmosphere. Following treatment with test compounds, such as a tyrphostin of interest, cells are washed with Ca²+ and Mg²+ free PBS and total protein is extracted as described (Haldar et al., Cell Death Diff. 1:109-115, 1994; Haldar et al., Nature 342:195-198, 1989; Haldar et al., Cancer Res. 54:2095-2097, 1994). In additional embodiments, serial dilutions of the test tyrphostin are used.

In some embodiments, phosphorylation is analyzed using Western blotting and immunodetection. These methods can be performed using Amersham ECL an enhanced chemiluminescence detection system and well known methodology. In one example, phosphorylation of cells can be carried out in phosphate free media (GIBCO) using 1 mCi/ml [P³²] orthophosphoric acid (NEN) for 6 hours in the presence of a test tyrphostin compound. Immunoprecipitation of P³² labeled cellular extract can be performed, for example, as described in Haldar et al., Nature 342:195-198, 1998. This immunoprecipitation utilizes an antibody that binds a substrate of interest. An immunocomplex is run on a 0.75 mm thick 10% SDS-PAGE. Subsequently, gels are dried and exposed for autoradiography.

In addition, phospho-amino acid analysis can be performed as is known in the art. For example, the analysis can be performed essentially as described in the manual for the Hunter thin layer electrophoresis system, HTLE700, (CBS Scientific Company Inc., USA). Briefly, P³² labeled immunoprecipitates are run on 10% SDS-PAGE gels. The immunoreactive bands of interest are cut out of the gel and eluted with 50 μM ammonium bicarbonate. After elution, the proteins are precipitated in the presence of 15%-20% TCA plus carrier protein, and washed with ethanol. Precipitated protein is then oxidized in performic acid and lyophilized. The dried pellet is resuspended in constant boiling HCl, heated at 110° C. and lyophilized. The residue is resuspended in pH 1.9 buffer (50 mcl formic acid, 156 mcl acetic acid, 1794 mcl H₂ O) containing phospho-amino acid standards and spotted on a PEI cellulose plate. Two-dimensional thin layer chromatography is run using the pH 1.9 buffer for the first dimension and pH 3.5 buffer (100 ml acetic acid, 10 ml pyridine, 1890 ml H₂ O) for the second. The plate is baked at 65° C. for 10 minutes, and the cold standards are visualized by spraying the plate with 0.25% ninhydrin and returning the plate to the 65° C. oven for 15 minutes. The plate is then exposed to film, such as to Kodak X-omat AR film, for two to four weeks.

Additional examples of methods that can readily be used to determine if a tyrphostin of interest activates ERK are described in the Examples section below. Any of these methods can be used to determine if a tyrphostin of interest activates ERK, such as by inducing phosphorylation of a substrate. In one embodiment, the ERK activation is compared to ERK activation in a control cell, such as a cell not contacted with the agent, or contacted with a media or a saline solution. However, ERK activation can also be determined by comparing the level of ERK activity as compared to the activity in a cell contacted with tyrphostin A9. One of skill in the art can readily determine a statistically test of use, such as, but not limited to, a student T test. Generally, a statistically significant result is found at p<0.05.

For administration to a subject, a therapeutically effective dose of a pharmaceutical composition containing the tyrphostin, or the pharmaceutically acceptable salt thereof, such as tyrphostin A9, can be included in a pharmaceutically acceptable carrier. The subject can be any subject of interest.

In one example, the pharmaceutical compositions are prepared and administered in dose units. Solid dose units are tablets, capsules and suppositories. For treatment of a subject, depending on activity of the compound, manner of administration, nature and severity of the disorder, age and body weight of the patient, different daily doses are necessary. Under certain circumstances, however, higher or lower daily doses may be appropriate. The administration of the daily dose can be carried out both by single administration in the form of an individual dose unit or else several smaller dose units and also by multiple administrations of subdivided doses at specific intervals.

The pharmaceutical compositions are in general administered topically, intravenously, orally, intracranially, intramuscularly, parenterally or as implants, but even rectal use is possible in principle. Suitable solid or liquid pharmaceutical preparation forms are, for example, granules, powders, tablets, coated tablets, (micro)capsules, suppositories, syrups, drops, emulsions, suspensions, creams, aerosols, drops or injectable solution in ampoule form and also preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of present methods for drug delivery, see Langer, Science 249:1527-1533, 1990.

The pharmaceutical compositions can be administered locally or systemically. In one embodiment, a therapeutically effective dose is the quantity of a tyrphostin that is sufficient to promote neurite outgrowth or nerve regeneration. In a specific example, a therapeutically effective amount is an amount sufficient to increase the neurite outgrowth and/or nerve regeneration induced by administration of nerve growth factor (NGF). In another specific example, a therapeutically effective amount is an amount necessary to prevent, to cure or at least partially arrest the symptoms of a neurodegenerative disorder and its complications. Amounts effective for this use will, of course, depend on the severity of the disease and the weight and general state of the patient, as well as the absorption, inactivation, and excretion rates of the therapeutically-active compound or component, the dosage schedule, and amount administered as well as other factors known to those of skill in the art. It also should be apparent to one skilled in the art that the exact dosage and frequency of administration will depend on the particular compounds administered, the particular condition being treated, the severity of the condition being treated, the age, weight, general physical condition of the particular patient, other medication the individual may be taking. Typically, dosages used in vitro may provide useful guidance in the amounts useful for in situ administration of the pharmaceutical composition, and animal models may be used to determine effective dosages for treatment of particular disorders. Various considerations are described, e.g., in Gilman et al., eds., Goodman And Gilman's: The Pharmacological Bases of Therapeutics, 8th ed., Pergamon Press, 1990; and Remington's Pharmaceutical Sciences, 17th ed., Mack Publishing Co., Easton, Pa., 1990, each of which is herein incorporated by reference.

In one example, the tyrphostin can be administered as a single dose per day, or it can be divided into at least two unit dosages for administration over a 24-hour period, or it may be a single continuous dose for a longer period of time, such as 1-10 weeks. Treatment may be continued as long as necessary to achieve the desired results. For instance, treatment may continue for about 3 or 4 weeks up to about 12-24 months. The tyrphostin can also be administered in several doses intermittently, such as every few days (for example, at least about every two, three, four five or ten days) or every few weeks (for example at least about every two, three, four, five or ten weeks).

The tyrphostin, such as tyrphostin A9 or pharmaceutical salt thereof, can be formulated into therapeutically-active pharmaceutical compositions that can be administered to a subject parenterally or orally. Parenteral administration routes include, but are not limited to, subcutaneous injections (subcutaneous (SQ) and depot SQ), intravenous (IV), intramuscular (IM and depot IM), intrasternal injection or infusion techniques, intranasal (inhalation), intrathecal, transdermal, topical, and ophthalmic.

The tyrphostin or pharmaceutically acceptable salt thereof, can be mixed or combined with a suitable pharmaceutically acceptable carrier to prepare pharmaceutical compositions. Pharmaceutically acceptable carriers include, but are not limited to, ion exchangers, alumina, aluminum stearate, lecithin, serum proteins (such as human serum album n), buffers (such as phosphates), glycine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat, for example. Liposomal suspensions may also be suitable as pharmaceutically acceptable carriers. Upon mixing or addition of the agent(s), the resulting mixture may be a solution, suspension, emulsion, or the like. These may be prepared according to methods known to those skilled in the art. The form of the resulting mixture depends upon a number of factors, including the intended mode of administration and the solubility of the agent in the selected carrier or vehicle. In one example, the effective concentration is sufficient for lessening or ameliorating at least one symptom of the disease, disorder, or condition treated and may be empirically determined.

Pharmaceutical carriers or vehicles suitable for administration of the tyrphostin or pharmaceutically acceptable salts thereof include any such carriers known to be suitable for the particular mode of administration. In addition, the active materials can also be mixed with other active materials that do not impair the desired action, or with materials that supplement the desired action, or have another action. The agents may be formulated as the sole pharmaceutically active ingredient in the composition or may be combined with other active ingredients.

Methods for solubilizing may be used where the agents exhibit insufficient solubility in a carrier. Such methods are known and include, but are not limited to, using cosolvents such as dimethylsulfoxide (DMSO), using surfactants such as Tween®, and dissolution in aqueous sodium bicarbonate. The tyrphostin can be prepared with carriers that protect them against rapid elimination from the body, such as time-release formulations or coatings. Such carriers include controlled release formulations, such as, but not limited to, microencapsulated delivery systems. The therapeutically-active tyrphostin is included in the pharmaceutically acceptable carrier in an amount sufficient to exert a therapeutically useful effect in the absence of undesirable side effects on the subject treated. The therapeutically effective concentration may be determined empirically by testing the compounds in known in vitro and in vivo model systems for the treated condition.

Injectable solutions or suspensions can be formulated, using suitable non-toxic, parenterally-acceptable diluents or solvents, such as mannitol; 1,3-butanediol; water; saline solution; Ringer's solution or isotonic sodium chloride solution; or suitable dispersing or wetting and suspending agents, such as sterile, bland, fixed oils, including synthetic mono- or diglycerides, and fatty acids, including oleic acid; a naturally occurring vegetable oil such as sesame oil, coconut oil, peanut oil, cottonseed oil, and the like; polyethylene glycol; glycerine; propylene glycol; or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required. Where administered intravenously, suitable carriers include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers.

For topical application, the extract fraction may be made up into a solution, suspension, cream, lotion, or ointment in a suitable aqueous or non-aqueous vehicle. Additives can also be included, e.g., buffers such as sodium metabisulphite or disodium edeate; preservatives such as bactericidal and fungicidal agents, including phenyl mercuric acetate or nitrate, benzalkonium chloride or chlorhexidine, and thickening agents, such as hypromellose.

If the tyrphostin is administered orally as a suspension, the pharmaceutical compositions can be prepared according to techniques well known in the art of pharmaceutical formulation and may contain microcrystalline cellulose for imparting bulk, alginic acid or sodium alginate as a suspending agent, methylcellulose as a viscosity enhancer, and sweeteners/flavoring agents. Oral liquid preparations can contain conventional additives such as suspending agents, e.g., sorbitol, syrup, methyl cellulose, glucose syrup, gelatin, hydrogenated edible fats, emulsifying agents, e.g., lecithin, sorbitan monooleate, or acacia; non-aqueous vehicles (including edible oils), e.g., almond oil, fractionated coconut oil, oily esters such as glycerine, propylene glycol, or ethyl alcohol; preservatives such as methyl or propyl p-hydroxybenzoate or sorbic acid, and, if desired, conventional flavoring or coloring agents. The pharmaceutical compositions also can be administered in the form of a tea. As immediate release tablets, these compositions can contain microcrystalline cellulose, dicalcium phosphate, starch, magnesium stearate and lactose and/or other excipients, binders, extenders, disintegrants, diluents and lubricants.

If oral administration is desired, the tyrphostin or pharmaceutically acceptable salt thereof can be provided in a composition that protects it from the acidic environment of the stomach. For example, the composition can be formulated in an enteric coating that maintains its integrity in the stomach and releases the active compound in the intestine. The composition can also be formulated in combination with an antacid or other such ingredient.

Oral compositions will generally include an inert diluent or an edible carrier and can be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition.

The tablets, pills, capsules, troches, and the like can contain any of the following ingredients or compounds of a similar nature: a binder such as, but not limited to, gum tragacanth, acacia, corn starch, sorbitol, polyvinylpyrrolidone or gelatin; a filler such as microcrystalline cellulose, starch, calcium phosphate, glycine or lactose; a disintegrating agent such as, but not limited to, alginic acid and corn starch; a lubricant such as, but not limited to, magnesium stearate, talc, polyethylene glycol, or silica; a gildant, such as, but not limited to, colloidal silicon dioxide; a sweetening agent such as sucrose or saccharin; disintegrants such as potato starch; and dispersing or wetting agents such as sodium lauryl sulfate; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose or glycerin as a sweetening agent and certain preservatives, dyes and colorings, and flavors.

When administered orally, the compounds can be administered in usual dosage forms for oral administration. These dosage forms include the usual solid unit dosage forms of tablets and capsules as well as liquid dosage forms such as solutions, suspensions, and elixirs. When the solid dosage forms are used, they can be of the sustained release type so that the compounds need to be administered only once or twice daily.

The tyrphostin, such as tyrphostin A9 optionally be co-administered with at least one other neurotrophic agent such as the neurotrophins (nerve growth factor (NGF) and NT-3), brain derived neurotrophic factor (BDNF), ciliary neurotrophic factor (CNTF), insulin growth factors (IGFs), FK506, an FKBP12-binding FK506 analog. In one example, the tyrphostin or pharmaceutically acceptable salt thereof is administered in conjunction with nerve growth factor. The administration can be simultaneous or sequential.

The tyrphostin, such as tyrphostin A9 optionally be co-administered with other agents. One of skill in the art can readily identify appropriate additional therapeutic agents. For example, for the treatment of multiple sclerosis, an immunosuppressive agent can be administered to the subject.

In one example, an agent such as beta interferon or an IL-2 receptor antagonist is administered in conjunction with the tyrphostin. In one specific non-limiting example, the agent is an antibody that specifically binds the IL-2 receptor. Antibodies that specifically bind the IL-2 receptor are known in the art (for example, see U.S. Pat. No. 5,011,684; U.S. Pat. No. 5,152,980; U.S. Pat. No. 5,336,489; U.S. Pat. No. 5,510,105; U.S. Pat. No. 5,571,507; U.S. Pat. No. 5,587,162; U.S. Pat. No. 5,607,675; U.S. Pat. No. 5,674,494; U.S. Pat. No. 5,916,559). Peptide antagonists of the IL-2 receptor, including antagonists of Tac (p55) and p75 (IL-2Rβ) are also known (for example, peptide antagonists for p55 and p75 are disclosed in U.S. Pat. No. 5,635,597). Other agents for treatment of MS can also be utilized, such as, but not limited to, T cell receptor peptides or estrogen.

The methods disclosed herein can be useful whenever neurite outgrowth is sought, for example following any acute or chronic nervous system injury resulting from physical transection/trauma, contusion/compression or surgical lesion, vascular pharmacologic insults including hemorrhagic or ischemic damage, or from neurodegenerative or other neurological diseases wherein neurite regeneration is desired.

In one embodiment, the subject has a neurodegenerative disorder such as Alzheimer's disease, Pantothenate kinase associated neurodegeneration, Parkinson disease, Huntington disease, human immunodeficiency virus (HIV) encephalopathy, multiple sclerosis (MS) or amyotrophic lateral sclerosis. In another embodiment, the subject has a “neurodegenerative-related disorder such as palilalia, tachylalia, echolalia, gait disturbance, perseverative movements, bradykinesia, spasticity, rigidity, retinopathy, optic atrophy, dysarthria, and dementia.

The methods can also be used in association with procedures such as a surgical nerve graft, or other implantation of neurological tissue, to promote healing of the graft or implant, and promote incorporation of the graft or implant into adjacent tissue. According to another aspect, the compositions could be coated or otherwise incorporated into a device or biomechanical structure designed to promote nerve regeneration.

In one embodiment, a transection of a peripheral nerve or a spinal cord injury can be treated by administering a nerve regenerative stimulating amount of the tyrphostin, such as tyrphostin A9, to a mammal and grafting to the peripheral nerve or spinal cord an allograft (Osawa et al., J. Neurocytol. 19:833-849, 1990; Buttemeyer et al., Ann. Plastic Surgery 35:396-401, 1995) or an artificial nerve graft (Madison and Archibald, Exp. Neurol. 128:266-275, 1994; Wells et al., Exp. Neurol. 146:395-402, 1997). The space between the transected ends of the peripheral nerve or spinal cord can be filled with a non-cellular gap-filling material such as collagen, methyl cellulose, etc., or cell suspensions that promote nerve cell growth, such as Schwann cells (Xu et al., J. Neurocytol. 26:1-16, 1997), olfactory cells, and sheathing cells (Li et al., Science 277:2000-2002, 1997). The tyrphostin can be included together with such cellular or non-cellular gap-filling materials.

In a further embodiment, the tyrphostin or pharmaceutically acceptable salt thereof is provided to the site of injury in a biocompatible, bioresorbable carrier capable of maintaining the compound at the site and, where necessary, means for directing axonal growth from the proximal to the distal ends of a severed neuron. For example, means for directing axonal growth can be required where nerve regeneration is to be induced over an extended distance, such as greater than 10 mm. Many carriers capable of providing these functions are envisioned. For example, useful carriers include substantially insoluble materials or viscous solutions prepared as disclosed herein comprising laminin, hyaluronic acid or collagen, or other suitable synthetic, biocompatible polymeric materials such as polylactic, polyglycolic or polybutyric acids and/or copolymers thereof. In one example, the carrier includes an extracellular matrix composition derived, for example, from mouse sarcoma cells.

In a certain example, the tyrphostin or pharmaceutically acceptable salt thereof is disposed in a nerve guidance channel which spans the distance of the damaged pathway. The channel acts both as a protective covering and a physical means for guiding growth of a neurite. Useful channels include a biocompatible membrane, which may be tubular in structure, having a dimension sufficient to span the gap in the nerve to be repaired, and having openings adapted to receive severed nerve ends. The membrane can be made of any biocompatible, nonirritating material, such as silicone or a biocompatible polymer, such as polyethylene or polyethylene vinyl acetate. The casing also may be composed of biocompatible, bioresorbable polymers, including, for example, collagen, hyaluronic acid, polylactic, polybutyric, and polyglycolic acids. In one embodiment, the outer surface of the channel is substantially impermeable.

The disclosure is illustrated by the following non-limiting Examples.

### EXAMPLES

### Example 1

### Material and Methods

Primary Neuronal Cultures: The substantia nigra or striatal region is dissected from 4 day-old Sprague-Dawley rats and incubated in minimum essential medium (MEM) containing 20 U/ml papain for 2 hours at 37° C. The tissue is then triturated using fire-polished Pasteur pipettes in MEM supplemented with 10% fetal bovine serum, 0.45% glucose, 5 pg/ml insulin, 0.5 mM glutamine, 100 U/ml penicillin, and 100 μg/ml streptomycin. Cells are plated on 18 mm diameter poly-D-lysine-coated glass coverslips (Fisher 12-545-84 18cir-1D) at a density of 75,000 cells per coverslip for immunofluorescence imaging or in 12-well or 6-well tissue culture plates for immunoblotting studies. Neurobasal medium (GibcoBRL, Carlsbad, Calif.) containing B27 supplement (GibcoBRL) and 500 nM L-glutamine is added one hour after initial plating. To obtain a near pure neuronal culture, cytosine arabinoside (AraC) 10 μM is added after 1 day of culturing and for 2 days to stop the proliferation of the astrocytes. After 3 days, the medium is changed to remove AraC. The cultures are maintained in a humidified atmosphere with 5% CO₂ for at least 10 days before experiments.

Cell preparation for microscopy: Neuroblastoma cells and rat striatal neurons are grown on glass coverslips and treated with poly-D-Lysine. Cells are fixed in 4% paraformaldehyde/Tris buffered saline (TBS, 50 mM Tris, 8% NaCl, pH 7.4) for 15 minutes, permeabilized and blocked in 5% goat serum and 0.5% Triton X-100 in TBS for 1 hour at room temperature. Cells are incubated with primary antibody (including rabbit anti-active ERK (p-ERK) antibody, 1/500 dilution, Promega, Madison, Wis.; rabbit anti-total ERK (t-ERK) antibody, 1/500 dilution, NEB, Beverly, Mass.; mouse anti-tyrosine hydroxylase (TH) antibody, 1:1000 dilution, Sigma, St Louis, Mo.; mouse anti-β-tubulin antibody, 1:1000 dilution, Sigma), washed, and incubated for 1 hour with Alexa 568 (red) or 488 (green)-labeled goat anti-rabbit or mouse IgG (1:500, Molecular Probes, Eugene, Oreg.). The coverslips are then mounted with the ProLong™ antifade kit (Molecular Probes) and dried in the dark. The samples are examined by an Olympus CX640 fluorescence microscope, or scanned with a Leica TCS SP confocal laser scanning microscope. Images are digitally captured and recorded with computer. The confocal images are deconvolved using Power HazeBuster™ imaging program (VayTek, Inc., Fairfield, Va.).

Cell stimulation, immunoblotting, and immunoprecipitation: Cells or neurons are grown in 6-well or 12-well plates to 80-90% confluence. The cells are starved in serum-free DMEM overnight, and then incubated with receptor agonists at 37° C. for the indicated durations. In some experiments, signaling inhibitors are added 45 minutes before simulation. Incubation is terminated by placing the tissue culture cluster on ice and rapidly aspirating the medium, followed by the addition of ice-cold RIPA⁺ buffer (50 mM Tris-HCl, pH 7.5, 150 mM NaCl, 1% NP-40, 0.5% deoxycholate, 0.1% SDS, 1 mM NaVO3, and protease inhibitor (Calbiochem, San Diego, Calif.)) and incubation for 15 minutes with shaking. After centrifugation (14,000×g at 4° C. for 15 minutes), the supernatant is collected and the protein concentration is measured and adjusted using RIPA⁺ buffer. Samples (30 μl) with equal amounts of protein mixed with Laemmli loading buffer are denatured at 70° C. for 10 minutes and separated by SDS-PAGE. Alternatively, for coprecipitation experiments solubilized cell lysates are centrifuged at 20,000×g for 15 minutes at 4° C., and the supernatants are collected and precleared with protein G-Sepharose beads (Sigma). Equal amounts of cellular proteins are incubated with primary antibody (including rabbit anti-PI3K, PDK1, and PKB/Akt antibodies, 1:500 dilution, NEB) for 2 hours at 4° C., followed by incubation with protein G-Sepharose for 1 hour. The resulting immune complexes are washed three times with lysis buffer and then resuspended in 40 μl of Laemmli loading buffer for separation by SDS-PAGE. The proteins are transferred to PVDF membrane (Millipore), detected with the primary antibodies (including anti-active ERK; anti-p-Tyr clone 4G10, Upstate; anti-TH; anti-DAT, sigma, anti-β-tubulin, etc.) and visualized with the SuperSignal™ West Pico chemiluminescent kit (Pierce Biotechnology, Rockford, Ill.). The intensity of bands is quantified using ImageQuant™ 5.2 (Amersham). When the phosphorylated form of a protein is detected, the blot is routinely stripped and re-probed with an antibody for total protein to ensure that equal amounts of protein are loaded.

Analysis of neurite length in SH-SY5Y neuroblastoma cells: SH-SY5Y neuroblastoma cells develop axonal-like processes on treatment with NGF. For analysis of process length, cells (20 fields/well) are randomly photographed at 96 and 168 hours. Neurite lengths are measured on photographic prints using a Houston Instrument HI-PAD digitizing tablet connected to a computer with appropriate software (Bioquant IV); only processes more than twice the cell body length are measured. Data from identically treated wells are combined. Mean values and histograms are constructed from these data; each histogram is constructed from measurement of 90 to 160 cells. Histograms are compared using a Mann-Whitney U test (α=0.05), which makes no assumptions about the shape of the distribution.

Dopamine uptake: NS20Y cells or neuron cultures are plated on poly-L-lysine (for NS20Y) or poly-D-lysine (for neuron culture)-coated 24-well plates and grown to confluence. The medium is decanted and cells are prepared for [³H]dopamine uptake. Cells are incubated in the presence or absence of mazindol (10 μM to determine nonspecific uptake) for at least 10 minutes prior to addition of [³H]dopamine. The uptake assay (final volume 0.5 ml) is initiated by the addition of 50 nM [³H]dopamine and 2 μM dopamine in Krebs-HEPES buffer. Pargyline (monoamine oxidase (MAO) inhibitor) and tropolone (catechol-O-methyl transferase (COMT) inhibitor) are included in the buffer to prevent metabolic degradation of dopamine. Assays are carried out at the 37° C. for 10 minutes. Experiments are terminated by aspiration and treatment with 250 μl of 0.1 M HCl. Radioactivity remaining in each well is determined using liquid scintillation spectrometry. Experiments will be conducted with triplicate determinations.

Tyrosine hydroxylase histoimmunostaining and quantitative morphology: On day 21 as described in Section D3, mice are euthanized via perfusion with fixative agent (2.5% glutaraldehyde, 0.5% paraformaldehyde, 0.1% picric acid, and 2 mM NaVO₃ in 0.1M HEPES buffer, pH 7.3) performed under anesthesia with mouse cocktail. The brains are processed for immunohistochemical studies. Sections (30 μm) are incubated with a monoclonal anti-tyrosine hydroxylase (TH, 1:1,000 dilution, Sigma) for 48 hours at 4° C. Biotinylated secondary antibodies followed by avidin-biotin complex are used. Immunoreactivity is visualized by incubation in 3,3′-diaminobenzidine/glucose/glucose oxidase. Total numbers of TH-positive neurons in SNc region are counted with a microcopy. Striatal OD of TH immunostaining, determined by the Scion Image program (Scion Corp., Frederick, Md., USA), is used as an index of striatal density of TH innervation.

Generation of siRNA knock-down in SH-SY5Y cells: Specific knock-down of gene expression in SH-SY5Y cells is achieved through use of the commercially validated siRNA sequences for human ERK1 (sc-29307, Santa Cruz Biotechnology, Santa Cruz, Calif.), ERK2 (sc-35335, Santa Cruz Biotechnology) and PI3K p85α (M-003020, Upstate, Charlottesville, Va.). The amount of siRNA used and the best time to measure the neurite outgrowth are empirically determined. The knock-down effect and time curves will first be determined by immunoblotting. The amount of siRNA is adjusted so that the peak knock-down time will fall at around 96 hours. Transfected SH-SY5Y cells are used to test for neurite elongation activity at the time points determined above.

Transfect neurons with HSV vectors: The HSV vectors are packaged. In experiments using primary neuronal cultures, a herpes simplex virus (HSV) vector is used to drive the expression of dominant negative mutants of target proteins. Rat SNc neurons are maintained at 37° C. in a humidified atmosphere with 5% CO2 in Neurobasal medium, B27 supplement, 50 μM of L-glutamine, 1% FBS, 1% HS, 0.05 U/ml penicillin, and 50 mg/ml streptomycin. Replication-defective HSV vectors are packaged at a titer of approximately 2×10⁵ infectious units/ml as described (Neve et al., Neuroscience 79:435-447, 1997). The amount of HSV used and the best time to stain or harvest the neurons are empirically determined.

Generation of dominant negative mutants: Dominant negative mutant of bovine p85α (dnp85) has been used successfully in rat cells to block the activation of PI3K (Amyere et al., Mol. Biol. Cell 11:3453-3467, 2000). The generation of a ddnp85 was reported before (Hara et al., Proc. Natl. Acad. Sci U.S.A 91:7415-741, 1994). Briefly, Two polymerase chain reaction fragments are amplified from bovine p85α cDNA nucleotides 988-1434 and nucleotides 1540-2175. These two fragments, together with a fragment of p85α (from 81 nucleotides upstream of ATG to nucleotide 1014, are cloned into pBluescript (Stratagene, La Jolla, Calif.) to generate dominant negative p85α with deletion of 35 amino acids from residues 479 to 513 of bovine p85α. Dominant-negative MEK1 (dnMEK1) with mutation of lysine 97 alanine has been used in numerous studies (Olszewska-Pazdrak et al., J. Biol. Chem. 279:1853-1860, 2004; Uehara et al., Brain Res. 790:284-292, 1998; Gu et al., Glycobiology 14:177-186, 2004) and is commercially available from Upstate Biotech. The cDNA of dnp85 and dnMEK1 are subcloned into the pHSVPrPUC amplicon, which has the HSV-1 origin of replication, the immediate early (IE) 4/5 promoter, the SV40 polyadenylation signal, and the HSV-1 “a” packaging site. Recombinant plasmids are packaged into virus particles in the packaging line 2-2, using an IE2 deletion mutant viral strain as helper virus, as described (Neve et al., Neuroscience 79:435-447, 1997).

### Example 2

### A9 Induced MEK and PI3K-Dependent Sustained ERK Activation in Neuronal Cells

ERK activation has been shown to promote the neuron differentiation and survival. In PC12 cells, neurotrophin stimulation induces a persistent activation of the ERK pathway that results in outgrowth of neurites and eventual cessation of cell division (Greene et al., Proc. Natl. Acad. Sci. U.S.A. 73:2424-2428, 1976; Heasley et al., Mol. Biol. Cell 3:545-553, 1992; York et al., Nature 392:622-626, 1998; Marshall et al., Cell 80:179-185, 1995; Huff et al., J. Cell Biol. 88:189-198, 1998). The activation of ERK occurs through phosphorylation of threonine and tyrosine at the sequence TpEYp by the upstream MAPK/ERK kinase (MEK). Thus, the ability of A9 in activating ERK can be tested by quantifying the abundance of dually phosphorylated ERKs using an antibody for phospho-ERKs through both immunoblotting and immunocytochemistry.

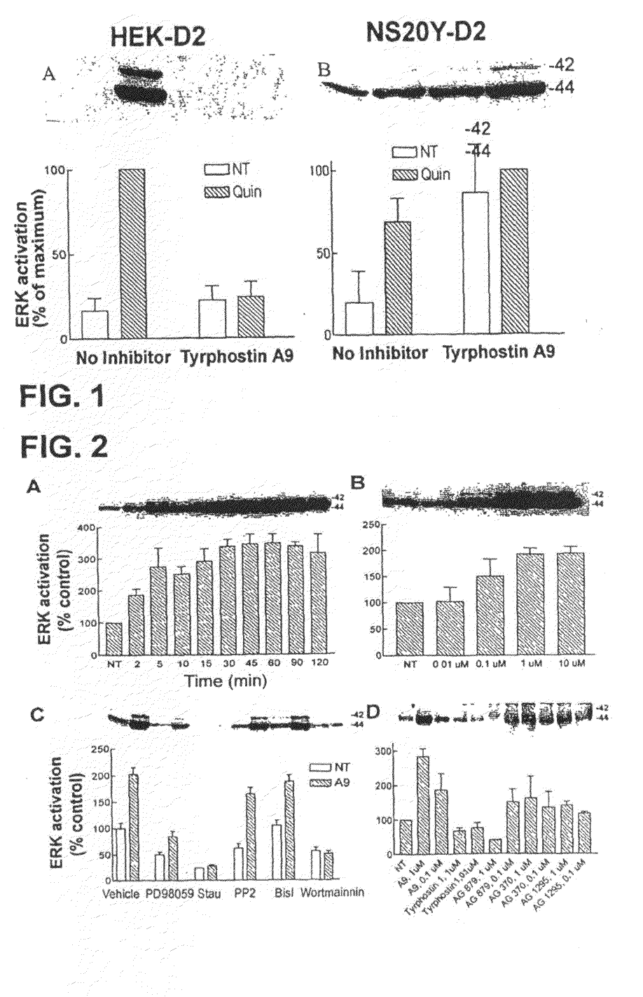

It was determined that A9 could activate ERK while characterizing the transactivation of receptor tyrosine kinases by the dopamine D2 receptor. Interestingly, although A9 pretreatment prevented D2 receptor-induced activation of ERK in HEK293 cells (a transformed cell line from human embryonic kidney cells), it caused robust activation of ERK on its own in NS20Y mouse neuroblastoma cells (FIG. 1).

A9-stimulated ERK activation was also observed in primary rat neostriatal neuron cultures but not in C6 cells, a glioma cell line. These results suggested that A9 stimulates ERK activation specifically in neuronal cells.

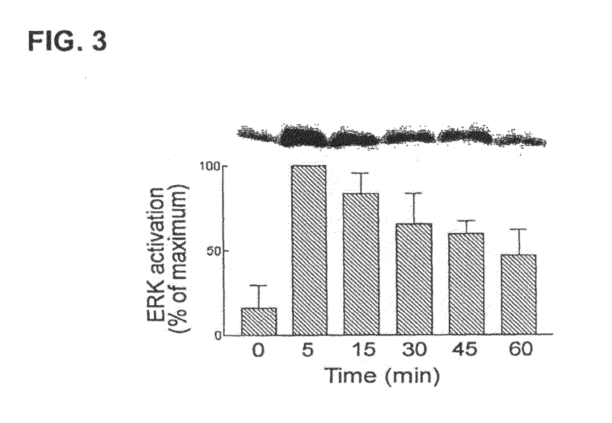

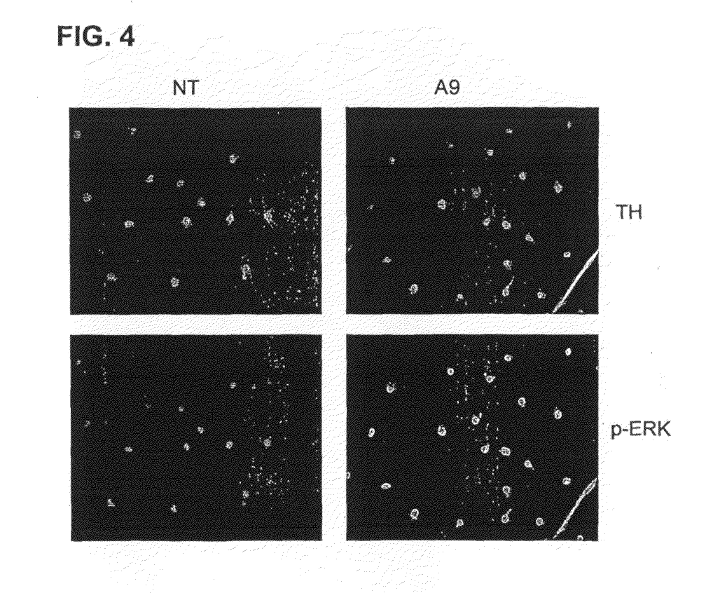

To further characterize ERK activation by A9 in NS20Y cells, its time and dose-response features were tested, as well as a potential mechanism. As shown in FIG. 2, A9 stimulated ERK activation in NS20Y cells in a time (FIG. 2A) and dose (FIG. 2B)-dependent manner. A9 activated ERK1 (p44) the most and had little stimulation on ERK2 (p42). The sustained nature of A9-stimulated ERK activation is in striking contrast to the short-lived quinpirole-induced ERK activation (FIG. 3). Pretreatment with the MEK inhibitor PD98059, the nonspecific serine/threonine protein kinase inhibitor stauosporine (Stau), and the PI3K inhibitor wortmannin prevented A9 from stimulating ERK activation (FIG. 2C), suggesting that A9 requires the activation of MEK, PI3K and an unknown serine/threonine protein kinase.

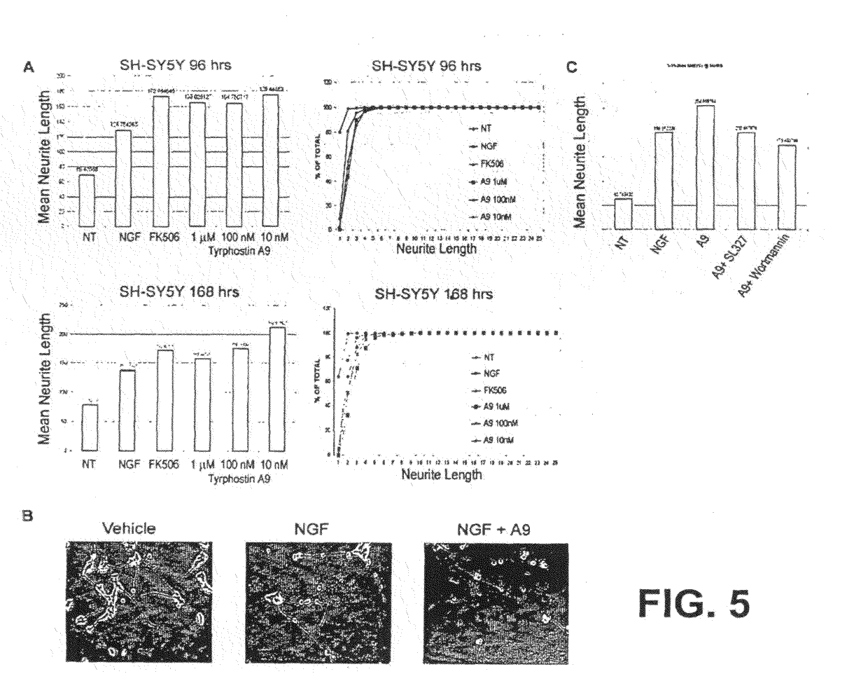

Interestingly, two other kinase inhibitors failed to prevent A9-stimulated ERK activity in the same cell line: the Src family protein tyrosine kinase inhibitor PP2, which was found by a previous study to block ERK activation induced by quinpirole in NS20Y cells, and the PKC (α, βI, βII, γ, δ, and ε) inhibitor bisindolylmaleimide I (BisI). It is noteworthy that BisI has little effect on the activation of PKCζ (IC50=5.8 μM) (71), a PKC subtype involved in the PI3K pathway. This effect of A9 is unrelated to its ability to inhibit the PDGF receptor because other PDGF receptor inhibitors, including AG1295 (FIG. 2D), AG82 and AG 23, did not stimulate ERK. Moreover, blocking PDGF receptor activity with the A9 analogs AG82 and AG23 did not prevent the effect of A9. EGF receptor inhibitor AG370 had moderate stimulation of ERK (FIG. 2D), as did the NGF receptor inhibitor AG879 at 0.1 μM (FIG. 1). But AG879 at 1 μM showed no effect, due possibly to the inhibition of endogenous NGF or toxicity. More importantly, in primary neuron culture established from the rat SNc, treatment with A9 for 1 hour induced robust ERK activation (FIG. 4). These data indicate that A9 induced robust and sustained ERK activation in NS20Y cells by a MEK- and PI3K-dependent mechanism.

### Example 3

### A9 Stimulated Neurite Outgrowth In Vitro

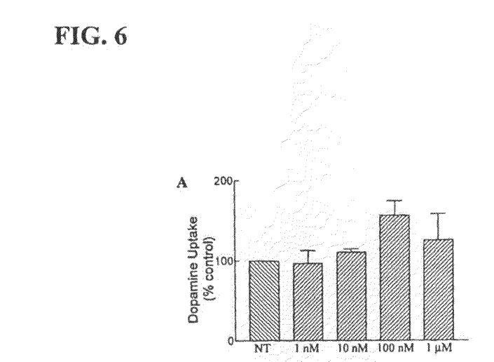

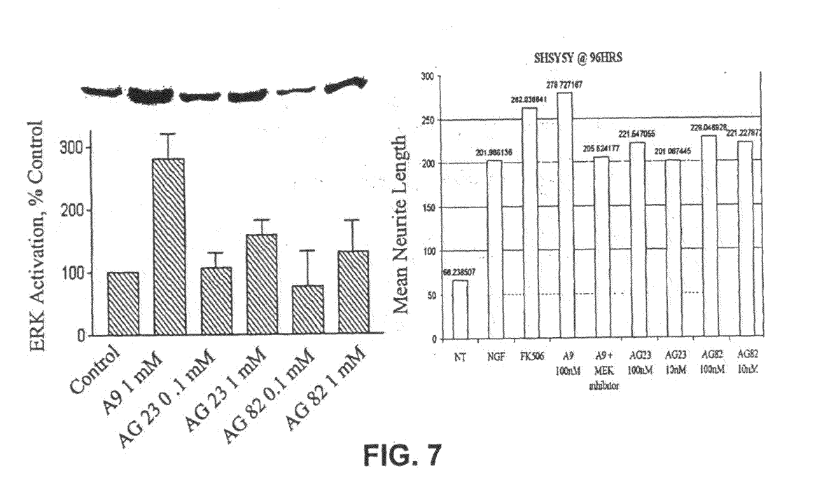

As disclosed herein, high and sustained ERK activation induces neurite growth in PC12 neuroblastoma cells and protects neuronal cells from neurotoxins. To test this, SH-SY5Y human neuroblastoma cells were used, which can be induced into neuron-like phenotype by NGF treatment. This is an established method for measuring neurite growth-promoting activity in vitro in Dr. Gold's lab (Gold et al., Neurosignals. 13:122-129, 2004; Gold et al., Exp. Neurol. 147:269-278, 1997). As shown in FIG. 5, SH-SY5Y cells developed long axonal-like processes upon exposure to NGF (10 ng/ml) that were dramatically increased in length by FK506 (100 nM, a positive control) and A9 at 10 nM, 100 nM and 1 μM concentrations at 96 and 168 hours time points. Representative micrographs at 168 hours for vehicle (control), NGF (10 ng/ml), and NGF (10 ng/ml) plus A9 (100 nM) are shown in FIG. 5B. Moreover, an experiment suggested that PD98059 (MEK inhibitor) did inhibit A9-induced neurite outgrowth in this cell line (FIG. 5C, see also FIG. 7).

### Example 4

### A9 Increased Dopamine Uptake and Stimulated Neuroprotection Against MPP⁺ in Dopamine Neurons

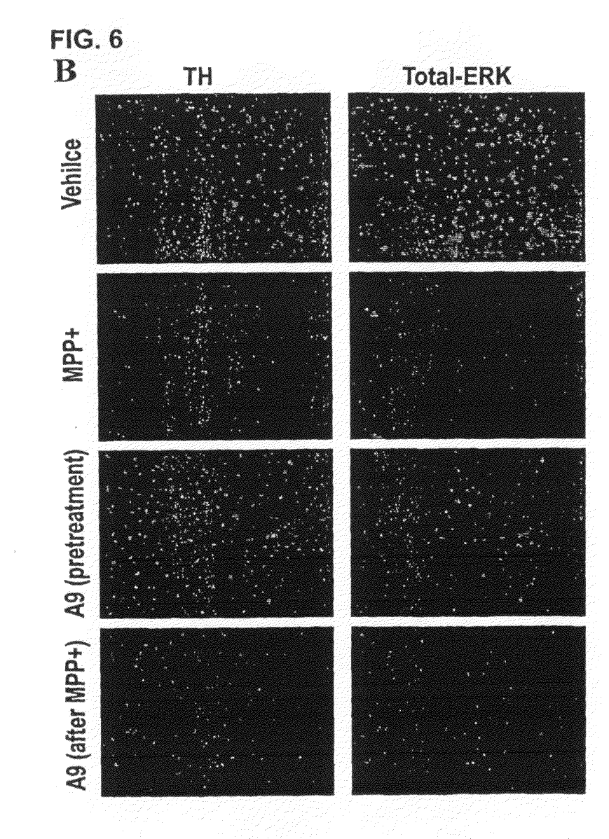

Dopamine uptake is used as one measure of the neurotrophic effect of A9 in dopaminergic neurons. By doing so, the complicated measurement of neurite length of neurons is avoided, that are usually individually diversified during in vitro culture. As shown in FIG. 6A, treatment with A9 for six days increased dopamine uptake in primary rat SNc neuron cultures in a concentration-dependent manner, with the maximum response reached at 100 nM. Most importantly, A9 treatment produced a robust protective effect against the neurotoxin MPP⁺ in primary dopaminergic neuron cultures (FIG. 6B). This protective effect of A9 is particularly striking considering that A9 also increased DAT activity/abundance, which would tend to increase MPP⁺ uptake and toxicity.

### Example 5

### Molecular Properties of Tyrphostin A9

The molecular structure of A9 is depicted above in the detailed description. To predict the likelihood of using A9 as a therapeutic drug, its molecular properties were calculated using the online service from the drug evaluation company Molinspiration Inc. (The program for this calculation is available on the internet at the Molinspiration website). The results show that A9 has the following molecular properties:

1. miLogP: Molinspiration LogP value (the partition coefficient in octanol/water) representing lipophilicity of a compound. This was calculated by the methodology developed by Molinspiration Cheminformatics Corp. as a sum of fragment-based contributions and correction factors. Compounds with miLogP greater than 5 might be difficult to dissolve in physiological buffers. The miLogP of A9 is 4.494.

2. TPSA: The molecular polar surface area, or TPSA, is calculated based on the methodology published by Ertl et al. (J. Med. Chem. 43:3714-3717, 2000) as a sum of fragment contributions. O- and N-centered polar fragments were considered. TPSA has been shown to be a very good descriptor characterizing drug absorption, including intestinal absorption, bioavailability, Caco-2 permeability and blood-brain barrier penetration. Brain penetration decreases with increasing TPSA. The TPSA of orally active drugs that are transported passively by the transcellular route should not exceed 120, while compounds with good BBB penetration generally have a TPSA of less than 70. The TPSA of A9 is 67.8.

3. MW: molecular weight. Smaller compounds (MW<500) usually have better bioavailability. The MW of A9 is 282.39.

4. nON and nOHNH: the number of hydrogen bond acceptors and donors. Too many hydrogen bond acceptor or donor groups impairs permeability across the cell membrane. Less than 10% of compounds entering Phase II clinical trials have nON or nOHNH greater than 10. The nON of A9 is 3 and the nOHNH of A9 is 1.

5. nrotb: number of rotatable bonds. This simple topological parameter is a measure of molecular flexibility. A rotatable bond is defined as any single non-ring bond, bound to nonterminal heavy (i.e., non-hydrogen) atom. Amine C—N bonds are not considered because of their high rotational energy barrier. A study suggested compounds that meet the two criteria of (a) 10 or fewer rotatable bonds and (b) 12 or fewer H-bond donors and acceptors will have a high probability of good oral bioavailability in the rat (Veber et al., J. Med. Chem. 45:2615-2623, 2002). The nRotb of A9 is 3.

6. Rule of 5: “Rule of 5” is a set of simple molecular descriptors used by Lipinski (Adv. Drug Deliv. Rev. 46:3-26, 2001). According to the “Rule of 5” most “drug-like” molecules have LogP≦5, molecular weight≦500, nON≦10, and nOHNH≦5. It is called the “rule of 5” because the upper limits are 5, 500, 2×5, and 5. Molecules violating more than one of these rules may have problems with bioavailability. A9 has no violation of the “rule of 5.”

5. Physical properties: A9 stock can be dissolved in ethanol, methylene chloride, or DMSO as high as 25 mg/ml. The stock of A9 can then be diluted in water or saline. The final concentration in saline can reach 10 mg/ml according to our experience. A9 is very stable in organic solvents and keeps its activity (indicated by its ability to stimulate ERK activation in NS20Y cells, data not shown) for 6 months at room temperature. Repeated freeze/thaw cycles in organic solvents do not affect its activity. In saline, its half-life was reported to be 4 days at 37° C. (Golumb, Atherosclerosis 125:171-182, 1996). The properties of A9 suggest good bioavailability as a therapeutic drug. A similar evaluation of the chemical nature of any tyrphostin can be used to determine if it is of use to induce neurite outgrowth and regeneration in a subject.

### Example 6

### A9-Induced Activation of ERK, PI3K, PDK1, and PKB/Akt

A9 treatment induced ERK activation in both NS20Y cells and in dopaminergic neurons. The use of MEK inhibitor PD98059 indicated that ERK is activated by A9 treatment in SH-SY5Y as well. SH-SY5Y cells are treated with A9, NGF (10 ng/nl), or EGF (10 ng/ml) for different durations, including 5, 15, 30, 60, and 120 minutes, to generate time-response curves for A9, NGF, and EGF. The level of ERK activation is measured by immunoblotting for the active (phosphorylated) ERK. The time-point with the maximum ERK response determined above is chosen for immunoprecipitation studies to assess the activation of PI3K, PDK1, and PKB/Akt. Cells are lysed with RIPA⁺ buffer and precipitated with polyclonal antibodies for PI3K, PDK1, and PKB/Akt, and then the presence of p-Tyr in the precipitates will be determined by immunoblotting with the specific p-Tyr monoclonal antibody 4G10 (Upstate). A9 causes sustained ERK activation in SH-SY5Y cells, similar to that induced by NGF. A9 stimulation increases the phosphorylation of PI3K, PDK1, and PKB/Akt.

### Example 7

### The Effect of Kinase Inhibitors and siRNA Gene Suppression on A9-Induced Neurite Outgrowth in SH-SY5Y Cells

ERK and PI3K inhibitors are tested on the neurite outgrowth stimulated by A9 in SH-SY5Y cells. SH-SY5Y cells will be treated with NGF (10 ng/ml), NGF (10 ng/ml) plus A9 (100 nM), and inhibitor groups containing NGF, A9, and inhibitors. After 24 hours of stimulation, PD98059 (ERK), SL327 (ERK), wortmannin (PI3K) or LY294002 (PI3K) are added into the respective inhibitor testing groups. After 168 hours of stimulation, the neurite length in each group is measured and analyzed. To test the siRNA gene suppression, cells are transfected with commercially validated siRNA sequences (from Upstate and Santa Cruz Biotechnologies) for human ERK1 and the p85α unit of PI3K 24 hours before stimulation. Because A9 has little effect on ERK2, the knocking-down of ERK2 is not planed. The amount of siRNA used is adjusted so that the peak knock-down time-point will fall around 96 hours (determined by Western Blot). Student t-test is applied to show whether differences among different treatment groups are statistically significant. ERK and PI3K inhibitors, or siRNA knock-down of the kinases, prevents A9-induced neurite outgrowth in SH-SY5Y cells.

### Example 8

### Mechanisms of A9-Induced Neuroprotection of Dopaminergic Neurons In Vitro

The dopaminergic neurons that have been cultured on poly-D-lysine treated coverslips or 12-well cell culture plates for 10 days are treated with vehicle or A9 at 1 nM, 10 nM, 100 nM, or 1 μM for 7 days. MPP⁺ at 20 μM (a concentration that has been shown to be effective in SNc neuron cultures) is added 1 day before or after the A9 treatment. Half of the medium is changed with fresh medium containing A9 at day 4. To test the involvement of ERK and PI3K, inhibitors (including PD98059, SL327, wortmannin, and LY294002) are added with A9. To confirm these findings with pharmacological inhibitors, dominant negative mutants are created of MEK1 (dnMEK1) and PI3K p85α unite (dnp85) and expressed in neuron cultures by infection with HSV 12 hours before A9 treatment. The amount of HSV used and the best time to do functional assays is determined. To count the TH-positive neurons, the neurons on coverslips are fixed, stained for both tyrosine hydroxylase (TH) and total-ERK (control) at the end of A9 treatment and imaged with a conventional or confocal fluorescence microscope. At the same time, a duplicate set of neurons in 12-well plate is incubated with [³H]dopamine to measure the level of dopamine uptake. Immunoblotting is used to detect TH and β-tubulin in cell lysates from the neurons.

TH-positive neurons and associated dopamine uptake is almost absent from MPP⁺-treated cultures in the absence of A9, while those treated with A9 either before or after MPP⁺ treatment show increased survival rates. Neurons with A9 added before MPP⁺ treatment survive better than those with A9 added after MPP⁺ treatment. Incubation with ERK or PI3K inhibitors blocks the ability of A9 to protect or rescue MPP⁺-treated neuron cultures.

### Example 9

### Assessment of Neuroprotection and Neuroregeneration, Side Effects, and ERK and PI3K Activation Induced by A9 in MPTP-Treated C57BL/6 Mice and Primates

The MPTP-treated animal model has provided the best available tool to date for pathogenesis studies and the assessment of efficacy of new therapeutic interventions of PD. Systemic administration of MPTP in animals replicates most of the clinical Parkinsonian symptoms and the biochemical and pathologic hallmarks of the disease, including the selective loss of dopaminergic neurons in the SNc and severe reductions in the concentrations of dopamine, noradrenaline and serotonin in the striatum. Thus, to predict the efficacy of A9 (or another tyrphostin disclosed herein) in treating dopaminergic neuron loss in PD patients, A9 (or the other tyrphostin) is tested in MPTP-treated C57BL/6 mice, a mouse model of PD (Sunstrom et al., Brain Res. 528:181-188, 1990). The activity of ERK in the SNc, striatum, and other brain regions is also be examined by immunohistochemistry methods. Possible side effects of A9 (or the other tyrphostin) are assessed by monitoring behavior abnormalities and histopathologic changes in the important organs of the mice.

For example, to assess the neuroprotective activity of A9, C57BL/6 mice are divided into 5 groups (4 mice/group), and receive different treatments as outlined in Table 1.

TABLE 1
Treatment strategies for different mice groups. All injections will be daily.

| Group | | | Day 10 to | |
|---|---|---|---|---|
| # | Day 1 | Day 2 to day 9 | day 17 | Day 21 |
| 1 | A9, i.p. 5 | MPTP, s.c., 30 mg/kg + | saline | euthanasia |
| | mg/kg | A9, i.p. 5 mg/kg | | |
| 2 | Saline | MPTP, s.c., 30 mg/kg | A9, i.p. 5 | euthanasia |
| | | | mg/kg | |
| 3 | saline | MPTP, s.c., 30 mg/kg | saline | euthanasia |
| 4 | A9, i.p. 5 | A9, i.p. 5 mg/kg | saline | euthanasia |
| | mg/kg | | | |
| 5 | saline | Saline | saline | euthanasia |

At day 21, the mice are euthanized via perfusion with fixative agent performed under anesthesia. The brain is sectioned and the number of TH-positive neurons in SNc, the dopaminergic nerve innervation density in striatum will be compared by immunohistochemistry staining for TH. ERK and PI3K activation are examined in some of the brain sections. The histopathologic changes in the SNc and striatum of mice are evaluated by routine Hematoxilin and Eosin (H&E) staining. Any behavior abnormalities and histopathologic changes in all brain areas, as well as livers, hearts, blood vessels, lunges, eyes, spleens, lymph nodes, muscles, etc. associated with A9 treatment are carefully monitored for the possible side effects of A9 in this strain of mice. Finally, the concentration of A9 distributed in the CNS is measured.

It has been shown that the MPTP-treatment procedure produces an almost complete destruction (i.e. ˜95-99%) of TH-positive nerves in C57BL/6 mice (unpublished result). Based on the in vitro effects of A9 and the observation that A9 can cause ERK activation in the brain, A9 induces regenerative sprouting from damaged nigrostriatal dopaminergic neurons and also reduces the loss of dopaminergic cell bodies. The density of TH staining in the SNc is sharply decreased in MPTP-treated mice, but is reversed by the A9 treatment. In the SNc, obvious histopathologic changes are seen by H&E staining in MPTP-only treated mice (which is alleviated by A9 treatment). For the first experiment, 5 mg/kg of A9 is used in vivo. Although A9 can inhibit the activity of PDGF receptor, it has not been shown to produce obvious side effects observed (Golomb et al., Atherosclerosis 125:171-182, 1996).

One effective experimental model of Parkinson's disease is the MPTP-treated primate (Jenner et al., Parkinsonism. Relat Disord. 9:131-137, 2003). These primates, when treated with MPTP, develop most Parkinsonian symptoms including bradykinesia, rigidity, and postural abnormalities. In addition, MPTP-treated primates are responsive to all commonly used anti-Parkinsonian agents and display treatment-associated motor complications such as dyskinesia, wearing-off, and on-off phenomena. Thus the MPTP-treated primate provides a useful model for preclinical testing of the anti-Parkinsonian activity of A9. Thus, similar studies can be conducted in primates, using methods known in the art.

### Example 10

### A9 Reduced Axonal Damage in Mice with Autoimmune Experimental Encephalomyelitis (EAE), Suggesting that A9 has the Ability to Cross the BBB

MS is believed to be an autoimmune disease that develops when pathogenic T cells cross the blood-brain barrier and cause damage to the myelin sheath in the CNS. It has become clear that axons are also injured in the CNS of MS patients as well as in EAE, as revealed by histological and advanced magnetic resonance spectroscopic imaging techniques. This axonal injury is also apparent in an animal model of multiple sclerosis, namely autoimmune experimental encephalomyelitis induced in SJL mice.

Active EAE was induced with PLP139-151 in SJL mice (see Encinas et al. Nature Genet. 21:158-160, 1999, for a description of this mouse model). These mice were treated with 1 μg of A9 or vehicle at the onset of EAE 52 days after immunization. A9 (or the vehicle control) was administered orally.

Following treatment, the thoracic spinal cords were fixed in 4% PFA, embodied in paraffin and sectioned for IHC. Sections were then stained with either SMI 312 (mouse antibody cocktail for total neurofilaments, which stains all the axons) or SMI 32 (monoclonal mouse antibody specific for non-phosphorylated neurofilaments, which stains the damaged axons only) and visualized by mouse ICH kit from Vector.

In this animal model, A9 reduced the axonal damage in the spinal cord. The results demonstrated that A9-treatment effectively reduced both the loss of normal axons and the number of damaged axons. This result also showed that A9 has the ability to cross the blood brain barrier after oral administration.

It will be apparent that the precise details of the methods or compositions described may be varied or modified without departing from the spirit of the described invention. We claim all such modifications and variations that fall within the scope and spirit of the claims below.

1. A method of promoting neurite outgrowth in a subject, comprising administering to the subject a therapeutically effective amount of a tyrphostin or a pharmaceutically acceptable salt thereof having a structure of: 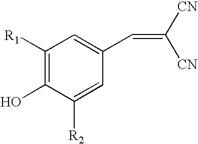
wherein R₁ and R₂ are a hydrogen (H) or a lower alkyl group, thereby promoting neurite outgrowth in the subject.

2. The method of claim 1, wherein at least one of R₁ and R₂ are a lower alkyl group.

3. The method of claim 1, wherein of R₁ and R₂ are a lower alkyl group.

4. The method of claim 1, wherein the tyrphostin is

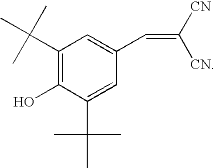

5. The method of claim 1, further comprising administering a therapeutically effective amount of nerve growth factor to the subject.

6. The method of claim 1, wherein the subject has a completely transected or a partially transected nerve.

7. The method of claim 6, wherein the subject has a completely transected or a partially transected peripheral nerve.

8. The method of claim 6, wherein the subject has a completely or partially transected spinal chord.

9. The method of claim 1, wherein the subject has a neurodegenerative disorder.

10. The method of claim 9, wherein the subject has multiple sclerosis.

11. A method of promoting neurite outgrowth, comprising
contacting a cell with an effective amount of a tyrphostin or a pharmaceutically acceptable salt thereof, having a structure of 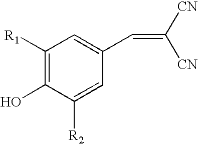
wherein R₁ and R₂ are a hydrogen (H) or a lower alkyl group, thereby promoting neurite outgrowth from the cell.

12. The method of claim 11, wherein at least one of R₁ and R₂ are a lower alkyl group.

13. The method of claim 11, wherein of R₁ and R₂ are a lower alkyl group.

14. The method of claim 11, wherein the tyrphostin is

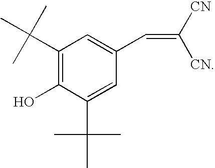

15. The method of claim 11, further comprising administering an effective amount of nerve growth factor to the cell.

16. The method of claim 11, wherein the cell is in vitro.

17. The method of claim 11, wherein the cell is in vivo.

18. A method of promoting nerve regeneration in a subject, comprising administering to the subject a therapeutically effective amount of a tyrphostin or a pharmaceutically acceptable salt thereof having a structure of: 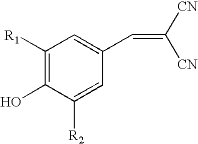
wherein R₁ and R₂ are a hydrogen (H) or a lower alkyl group, thereby promoting nerve regeneration in the subject.

19. The method of claim 18, wherein at least one of R₁ and R₂ are a lower alkyl group.

20. The method of claim 18, wherein of R₁ and R₂ are a lower alkyl group.

21. The method of claim 18, wherein the tyrphostin is

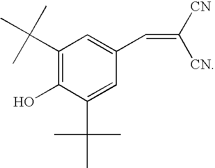

22. The method of claim 18, further comprising administering a therapeutically effective amount of nerve growth factor to the subject.

23. The method of claim 18, wherein the subject has a completely transected or a partially transected nerve.

24. The method of claim 21, wherein the subject has a completely transected or a partially transected peripheral nerve.

25. The method of claim 18, wherein the subject has a completely or partially transected spinal chord.

26. The method of claim 18, wherein the subject has a neurodegenerative disorder.

27. The method of claim 18, wherein the neurodegenerative disorder is multiple sclerosis.
